(19) United States
(12) Patent Application Publication
(10) Pub. No.: US 2025/0171865 A1
(43) Pub. Date: May 29, 2025

(54) CAPILLARY ELECTROPHORESIS METHODS FOR CHARACTERIZING GENOME INTEGRITY

(71) Applicant: DH TECHNOLOGIES DEVELOPMENT PTE. LTD., Singapore (SG)

(72) Inventors: Elliott Jones, Redwood City, CA (US); Tingting Li, Redwood City, CA (US); Jane Luo, Redwood City, CA (US); Sahana Mollah, Redwood City, CA (US); Mario Pulido, Redwood City, CA (US); Zhichang Yang, Redwood City, CA (US)

(21) Appl. No.: 18/715,638

(22) PCT Filed: Dec. 6, 2022
(86) PCT No.: PCT/IB2022/061851

§ 371 Date: May 31, 2024

Related U.S. Application Data
(60) Provisional application No. 63/287,266, filed on Dec. 8, 2021.

(51) Int. Cl. C12Q 1/70; C12Q 1/6806; C12Q 1/6825; G01N 27/447

(52) U.S. Cl. CPC C12Q 1/701; C12Q 1/6825; G01N 27/44726; G01N 27/44747; G01N 27/44782; G01N 27/44791; C12Q 1/6806

(57) ABSTRACT

Disclosed herein are methods for analyzing biological samples using capillary electrophoresis, including the characterization of genome integrity, assessment of genomic integrity and the sequencing of a nucleic acid genome, such as an RNA genome. Kits for characterizing genome integrity are also disclosed.

### RELATED APPLICATIONS

The present patent application claims the priority benefit of U.S. Provisional Patent Application Ser. No. 63/287,266, filed Dec. 8, 2021, the content of which is hereby incorporated by reference in its entirety into this disclosure.

### BACKGROUND

There are various challenges during the production of biomolecules, such as viral species production, including a limited ability to properly characterize an active virus product. Manufacturers and users do not have rapid, easy, and reliable methods to determine if there are unintentional products in a biological sample, including nucleic acid impurities outside of a viral species, and impurities, such as partial genome inside the viral species (defective interfering particles). Manufacturers and users also do not have a test with high precision and good dynamic range for viral species titer determination. Accurate titer is critical for obtaining dosage to response relationship of gene therapy treatments using viral species, such as lentivirus. Additionally, manufacturers and users do not have a fast, inexpensive test with low sample volume to determine the percent of full capsids in their viral species products.

With the demand for viral vectors, such as lentiviral vectors growing, so are the need to characterize these vectors better to ensure their safety and efficacy. Many methods were established for titer determination, but none has been developed to analyze the integrity of the RNA genome within the lentiviral vectors. Reverse transcription-PCR (RT-PCR) is a commonly used technique whereby RNA is isolated and then cDNA is generated using reverse transcription. The PCR is then used to amplify the nucleic acids of interest. While RT-PCR can detect the presence of short fragments on the RNA genome, but it cannot detect deletion mutants (e.g., a part of a chromosome or a sequence of DNA is left out during DNA replication) or impurities. In addition, both RT and PCR amplification are known to cause variability.

### SUMMARY

The inventors have recognized the need for high precision analysis of biological samples, including viral species. The claimed and described capillary electrophoresis methods offers high resolution and provides accurate size analysis that directly confirms the correct size of either the intact viral species, including intact lentivirus RNA genome or partial genome, and detect impurities. The claimed and described capillary electrophoresis methods also provide high sensitivity by using a laser-induced fluorescence detector, which enables detection of RNA genome without the amplification by required by RT-PCR. This method decreases the run and analysis time and also results in less errors. Additionally, the claimed and described capillary electrophoresis methods may be used with sample volumes smaller than what is required for RT-PCR.

An aspect of the disclosure relates to an analysis of biological samples, including the characterization of genome integrity with high sensitivity and high resolution. Results from genome integrity analysis of these biological samples can be useful in titer determination and determination of percent of full capsids in viral products. Another aspect of the disclosure includes the assessment of genomic integrity and the sequencing of a nucleic acid genome, such as an RNA genome.

One aspect of the disclosure relates to a method for characterizing genome integrity comprising:
incubating at least one biomolecule comprising nucleic acids with a chaotropic agent;
extracting the nucleic acids from the biomolecule;
diluting the nucleic acids;
heating the nucleic acids to denature the nucleic acids;
loading the denatured nucleic acids onto a capillary electrophoresis (CE) capillary, wherein the CE capillary is filled with a buffer comprising a polymer matrix;
applying a separation voltage to the CE capillary to separate the denatured nucleic acids; and
detecting at least one nucleic acid genome separated from the denatured nucleic acids with a detector.

In some aspects of the method, the biomolecule is a viral sample.

In some aspects of the method, the viral sample is a viral vector.

In some aspects of the method, the viral vector includes a nucleic acid genome, plasmid fragment contamination, and/or DNA fragment contamination from a host cell DNA.

In some aspects of the method, the viral vector is a retrovirus.

In some aspects of the method, the viral vector is a lentivirus.

In some aspects of the method, the lentivirus is a recombinant lentivirus.

In some aspects of the method, the at least one nucleic acid genome comprises deoxyribonucleic acid (DNA), ribonucleic acid (RNA), single stranded DNA (SSDNA), microRNA (miRNA), and/or messenger RNA (mRNA).

In some aspects of the method, the method further includes incubating the biomolecule with a carrier molecule.

In some aspects of the method, the carrier molecule is a poly (A) carrier, a poly (T) carrier, a poly (AT) carrier, glycogen, carrier RNA, or RNA from yeast.

In some aspects of the method, prior to incubating the biomolecule, the biomolecule is treated with an enzyme.

In some aspects of the method, the enzyme is selected from ribonuclease, deoxyribonuclease, endonuclease, and combinations thereof. In some aspects, the enzyme is a digestion enzyme.

In some aspects of the method, the chaotropic agent is a denaturing agent or lysis buffer.

In some aspects of the method, the chaotropic agent is guanidinium thiocyanate, guanidinium isothiocyanate, n-butanol, ethanol, guanidinium chloride, guanidinium hydrochloride, lithium acetate, magnesium chloride, 2-propanol, sodium dodecyl sulfate, thiourea, urea, sodium iodide, sodium perchlorate, potassium iodide, or combinations thereof.

In some aspects of the method, the nucleic acids are extracted using solid phase extraction, liquid-liquid extraction, a trap-and-elute workflow, filtration, organic solvent extraction or magnetic based purification.

In some aspects of the method, the solid phase extraction is spin column-based purification.

In some aspects of the method, the nucleic acids are eluted from the spin column using water.

In some aspects of the method, the nucleic acids are diluted using a sample solution, water, or combinations thereof.

In some aspects of the method, the sample solution is a sample loading solution.

In some aspects of the method, the sample solution is formamide.

In some aspects of the method, the water is deionized water or nuclease-free water.

In some aspects of the method, the nucleic acids are heated at a temperature between about 40°° C. to about 90° C., alternatively at a temperature between about 45°° C. to about 85° C., alternatively at a temperature between about 50°° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68°° C. to about 74° C., alternatively at a temperature between about 69° C. to about 73° C., alternatively at a temperature of about 70° C.

In some aspects of the method, the nucleic acids are heated for at least about 30 seconds, alternatively at least about 1 minute, alternatively at least about 90 seconds, alternatively at least about 2 minutes, alternatively at least about 3 minutes, alternatively at least about 4 minutes, alternatively at least about 5 minutes, alternatively at least about 6 minutes, alternatively at least about 7 minutes, alternatively at least about 8 minutes, alternatively at least about 9 minutes, alternatively at least about 10 minutes.

In some aspects of the method, the nucleic acids are cooled immediately after heating.

In some aspects of the method, the nucleic acids are cooled for at least about 5 minutes, alternatively at least about 6 minutes, alternatively at least about 7 minutes, alternatively at least about 8 minutes, alternatively at least about 9 minutes, alternatively at least about 10 minutes, alternatively at least about 15 minutes, alternatively at least about 20 minutes, alternatively at least about 25 minutes, alternatively at least about 30 minutes, alternatively at least about 35 minutes, alternatively at least about 40 minutes, alternatively at least about 45 minutes, alternatively at least about 50 minutes, alternatively at least about 55 minutes, alternatively at least about 60 minutes.

In some aspects of the method, the concentration of denatured nucleic acids loaded onto the CE capillary is at least about 0.25 ng/mL, alternatively at least about 0.28 ng/mL, alternatively at least about 0.33 ng/ml, alternatively at least about 0.36 ng/ml, alternatively at least about 0.39 ng/ml.

In some aspects of the method, the polymer matrix is selected from the group including crosslinked polymer, linear polymers, slightly branched polymers, linear polyacrylamide, polyethylene oxide, polyethylene glycol, dextran, and polyvinylpyrrolidone.

In some aspects of the method, the polymer matrix includes a fluorescent dye.

In some aspects of the method, the fluorescent dye is selected from the group including a cyanine-based dye, SYBR Green II, SYBR gold, SYBR Green I, LIFluor EnhanceCE, and Gel Green.

In some aspects of the method, the nucleic acids are separated using capillary zone electrophoresis, capillary gel electrophoresis, capillary isoelectric focusing, micellar electrokinetic capillary chromatography, or capillary electrochromatography.

In some aspects of the method, the detector is a UV detector or fluorescence detector.

In some aspects of the method, the detector is a laser-induced fluorescence (LIF) detector, a lamp-based fluorescence detector, or a native fluorescence detector.

In some aspects of the method, the method is used in an amplification-free workflow, a high-throughput screening application or a rapid screening workflow.

In some aspects of the method, the method is used in a multi-capillary electrophoresis system workflow.

In some aspects of the method, the multi-capillary electrophoresis system workflow comprises the use of a cartridge comprising at least two capillaries, alternatively at least three capillaries, alternatively at least four capillaries, alternatively at least five capillaries, alternatively at least six capillaries, alternatively at least seven capillaries, alternatively at least eight capillaries.

In some aspects of the method, the multi-capillary electrophoresis system workflow is used from the simultaneous analysis of nucleic acids from at least two biomolecules, alternatively at least three biomolecules, alternatively at least four biomolecules, alternatively at least five biomolecules, alternatively at least six biomolecules, alternatively at least seven biomolecules, alternatively at least eight biomolecules.

In some aspects of the method, at least two of the biomolecules have nucleic acids with different genome sizes. In some aspect, the method is used for titer determination of a viral product and/or determination of percent of full capsids in viral product or complete lentiviral particles

In a further aspect, the disclosure provides a kit for characterizing genome integrity, wherein the kit includes:
a chaotropic agent,
a CE capillary or a cartridge comprising at least two capillaries,
a buffer comprising a polymer matrix,
at least one sample solution, and
instructions for use.

In some aspects of the kit, the chaotropic agent is guanidinium thiocyanate, guanidinium isothiocyanate, n-butanol, ethanol, guanidinium chloride, guanidinium hydrochloride, lithium acetate, magnesium chloride, 2-propanol, sodium dodecyl sulfate, thiourea, urea, sodium iodide, sodium perchlorate, potassium iodide, or combinations thereof.

In some aspects of the kit, the kit further includes a carrier molecule.

In some aspects of the kit, the carrier molecule is a poly (A) carrier, a poly (T) carrier, a poly (AT) carrier, carrier RNA, glycogen, or RNA from yeast.

In some aspects of the kit, the kit further includes a solid phase extraction cartridge.

In some aspects of the kit, the solid phase extraction cartridge is a spin column.

In some aspects of the kit, the sample solution is formamide.

In some aspects of the kit, the kit further includes a digestion enzyme.

In some aspects of the kit, the digestion enzyme is selected from the group consisting of ribonuclease, deoxyribonuclease, endonuclease, and combinations thereof.

Other aspects and features of the present disclosure will become apparent to those ordinarily skilled in the art upon review of the following description of specific aspects of the disclosure in conjunction with the accompanying figures.

### BRIEF DESCRIPTION OF THE DRAWINGS

Aspects of the present disclosure will now be described, by way of example only, with reference to the attached Figures.

FIG. 1 illustrates a workflow for lentiviral vector genome integrity analysis according to an aspect of the disclosure.

FIG. 2 illustrates a lentivirus structure.

FIG. 3 illustrates a genome integrity analysis for four lentivirus vectors with different genome sizes.

FIG. 4 is an example of a sample plate layout and a reagent plate layout for running 16 samples in two columns.

FIG. 5 is an example of settings for a cartridge conditioning method.

FIG. 6 is an example of settings for a sample separation method.

FIG. 7 is an example of settings for the shutdown method.

FIG. 8 illustrates a comparison of RNA extraction with and without carrier RNA.

FIG. 9 illustrates a comparison of eluting RNA with nuclease free water or with 10% elution buffer (EB).

FIGS. 10A and 10B illustrate the effects of enzyme treatments on RNA profile of lentivirus samples.

FIGS. 11A and 11B illustrate a repeatability test for sample separation.

### DETAILED DESCRIPTION

It is to be understood that this disclosure is not limited to the particular methodology, protocols, and reagents described herein and as such may vary. It is also to be understood that the terminology used herein is for the purpose of describing particular aspects only and is not intended to limit the scope of the present disclosure or the appended claims.

Unless otherwise defined, all technical and scientific terms used herein have the same meaning as commonly understood by one of ordinary skill in the art to which the methods described herein belong.

The singular form “a”, “an” and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “exemplary” means serving as a non-limiting example, instance, or illustration. As utilized herein, the terms “e.g.,” and “for example” set off lists of one or more non-limiting aspects, examples, instances, or illustrations.

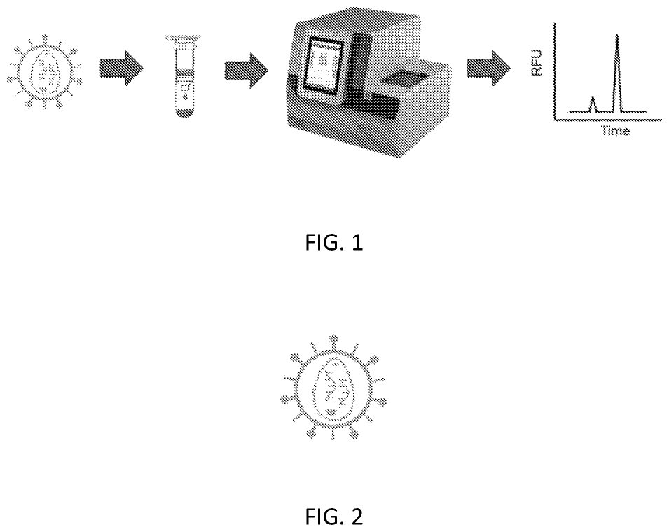

Aspects of this disclosure include methods for characterizing genome integrity by using a capillary electrophoresis platform (depicted in FIG. 1). Aspects of this disclosure can be used for amplification-free genome integrity analysis. The disclosed methods also achieve high separation resolution between intact viral vector nucleic acid genomes, partial genomes and impurities. In addition, the methods allow for multi-capillary separation and the simultaneous analysis of multiple biological samples with different genome sizes.

In an aspect of the disclosure, genome integrity is characterized by incubating at least one biomolecule comprising nucleic acids with a chaotropic agent. Non-limiting examples of nucleic acids include deoxyribonucleic acid (DNA), ribonucleic acid (RNA), single-stranded DNA (ssDNA), microRNA (miRNA), and messenger RNA (mRNA).

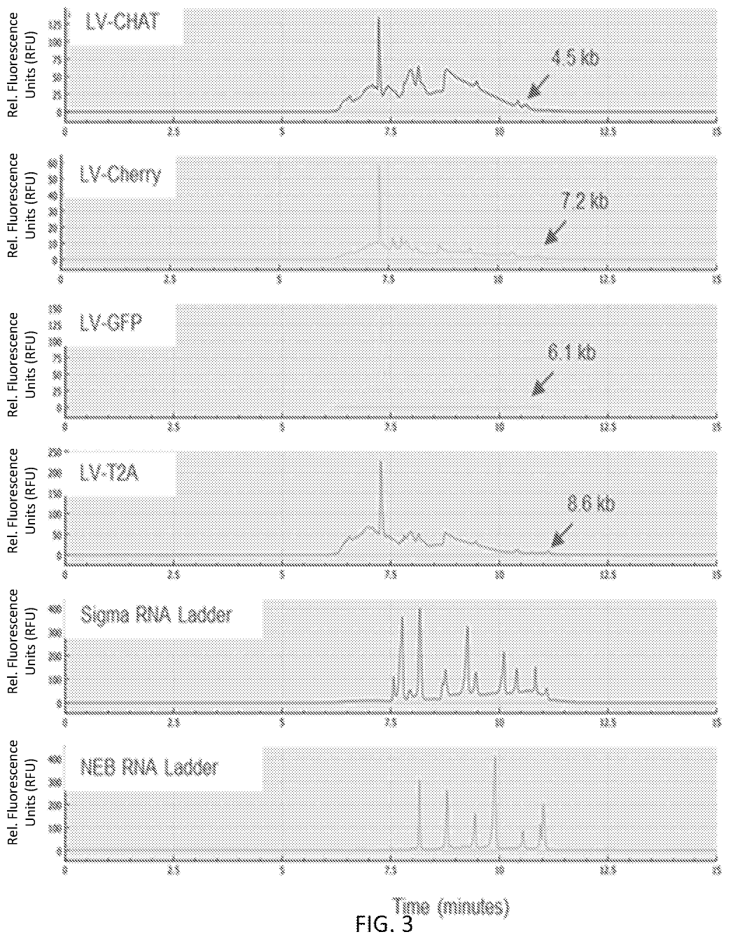

In some aspects, the biomolecule may be a viral sample, such as a viral vector. A non-limiting example of the viral vector or viral plasmid includes retrovirus or lentivirus. Lentivirus (depicted in FIG. 2) can integrate large DNA molecules into host cells, thus representing one of the most efficient gene delivery vehicles for transduction. Cockrell, A. S. and T. Kafri, Gene delivery by lentivirus vectors. Mol Biotechnol, 2007. 36(3): p. 184-204. For this particular application, lentivirus has several advantages, including its ability to infect non-dividing and dividing cells. For clinical therapeutic applications, the stable and long-term transgene expression of these retroviruses represents a great advantage. Anguela, X. M. and K. A. High, Entering the Modern Era of Gene Therapy. Annu Rev Med, 2019. 70: p. 273-288. The proteome of the lentivirus comprises five important structural and several non-structural proteins. The vital structural proteins are gp120 surface envelope protein (120 kDa), gp41 transmembrane envelope protein (41 kDa), p24 capsid protein (24 kDa), p17 matrix protein (17 kDa), and the p7/P9 nucleocapsid protein (7-11 kDa). The two envelope proteins of gp120 and gp41 are highly glycosylated. This post-translational modification (PTM) conceals important antigenic sites, thus helping the virus avoid recognition by the host's immune system. The p24 protein is the building block of the lentivirus capsid, and approximately 2000 of them assemble the full capsid.

The lentivirus (or lentivirus vector or “LV”) may also be a recombinant lentivirus. Recombinant lentivirus may be engineered to achieve expression of a certain gene. For example, the lentivirus may be engineered to express green fluorescent protein (GFP) under the CAG promoter (LV-CAG-GFP), express GFP under the human ChAT promoter (LV-ChAT-GFP), express Cre recombinase fusion with mCherry under the CAG promoter (LV-CAG-Cre-mCherry), express GFP and firefly luciferase (fLuc) via T2A linker under the EF1α promoter with co-expression of puromycin (LV-EF1α-GFP-T2A-fLuc-Puro). The lentivirus may be a Human CHAT lentivirus vector (LV-CHAT), a lentivirus vector expressing Cherry (LV-Cherry), a lentivirus vector expressing GFP (LV-GPF), a lentivirus vector expressing T2A (LV-T2A). In an aspect, the method may include a sizing standard such as a DNA or RNA ladder.

“Chaotrope” or “chaotropic agent” as used herein refers to a cosolute or compound that can disrupt hydrogen bonds. In one aspect of the disclosure, the chaotropic agent disrupts the structure of the biomolecule. A chaotropic agent may be, for example, a denaturing agent or lysis buffer. Other non-limiting examples of a chaotropic agent include guanidinium thiocyanate, guanidinium isothiocyanate, n-butanol, ethanol, guanidinium chloride, guanidinium hydrochloride, lithium acetate, magnesium chloride, 2-propanol, sodium dodecyl sulfate, thiourea, urea, sodium iodide, sodium perchlorate, potassium iodide, or combinations thereof.

In some aspects, the biomolecule is also incubated with a carrier molecule. In some aspects, carrier molecules are used to increase the yield of nucleic acid extracted. The carrier molecule may be a carrier nucleic acid or another molecule that mimics nucleic acid. Non-limiting examples of carrier molecules include a poly (A) carrier, a poly (T) carrier, a poly (AT) carrier, glycogen, or RNA from yeast.

In some aspects of the disclosure, it is desirable to remove any nucleic acid impurities in the biomolecule that may result from, for example, host cells and culture media. To remove nucleic acid impurities outside of the biomolecule, the biomolecule may be treated with an enzyme. Non-limiting examples of the enzyme include ribonuclease, deoxyribonuclease, endonuclease, and combinations thereof.

After incubating the biomolecule with the chaotropic agent, the nucleic acids are extracted from the biomolecule. The extraction can be carried out using solid-phase extraction, liquid-liquid extraction, a trap-and-elute workflow, filtration, organic solvent extraction or magnetic-based purification, or any other extraction techniques known in the art. In some aspects, the nucleic acids are extracted using spin column-based purification.

In an aspect, the nucleic acids are diluted prior to further analysis. The nucleic acids may be diluted with, for example, a sample solution, water, or a combination thereof. The sample solution may be a sample loading solution, and in some aspects, the sample solution is formamide. The water may be deionized water or nuclease-free water.

The nucleic acids may be heated, which further destabilizes or denatures the nucleic acids. In non-limiting examples, the nucleic acids are heated at a temperature between about 40° C. to about 90° C., alternatively at a temperature between about 45°° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C. The nucleic acids may also be heated for at least about 30 seconds, alternatively at least about 1 minute, alternatively at least about 90 seconds, alternatively at least about 2 minutes. After heating, in some aspects, the nucleic acids are cooled immediately. The nucleic acids may be cooled for at least about 1 minute, alternatively at least about 2 minutes, alternatively at least about 3 minutes, alternatively at least about 4 minutes, alternatively at least about 5 minutes.

In an aspect, the nucleic acids are loaded onto a capillary electrophoresis (CE) capillary. As used herein, “capillary” refers to a channel, tube, or other structure capable of supporting a volume of separation medium for performing electrophoresis. Capillary geometry can vary and includes structures having circular, rectangular, or square cross-sections, channels, groves, plates, etc. that can be fabricated by technologies known in the art. Capillaries of the present disclosure can be made of materials such as, but not limited to, silica, fused silica, quartz, silicate-based glass such as borosilicate glass, phosphate glass, or alumina-containing glass, and other silica-like materials. In some aspects, the methods can be adapted and used in any generally known electrophoresis platform such as, for example, electrophoresis devices comprising single or multiple microfluidic channels, etched microfluidic capillaries, as well as slab gel and thin-plate gel electrophoresis.

The CE capillary may be filled with a buffer comprising a polymer matrix or gel buffer prior to applying a separation voltage and/or loading the nucleic acid. In some aspects, the buffer comprising a polymer matrix or gel buffer is placed into a buffer vial. These buffer vials may be placed into buffer trays. In some aspects, the buffer comprising a polymer matrix or gel buffer may comprise additional components to facilitate the separation of the nucleic acids. Non-limiting examples of a suitable polymer matrix include crosslinked polymer, linear polymers, slightly branched polymers, linear polyacrylamide, polyethylene oxide, polyethylene glycol, dextran, and polyvinylpyrrolidone.

In some aspects, a fluorescent dye is added to the polymer matrix, the buffer, or both the polymer matrix and the buffer. The fluorescent dyes include, but are not limited to cyanine-based dye, such as Cy2, Cy3, Cy3B, Cy3.5, Cy5, Cy5,5, and Cy7, SYBR Green I, SYBR Green II, SYBR Gold, LIFluor EnhanceCE, Gel Green, PicoGreen, Thiazole orange, and Oxazole yellow.

In an aspect, a separation voltage is applied to the CE capillary, and the nucleic acids are moved towards a detector. The nucleic acids may be separated using capillary zone electrophoresis, capillary gel electrophoresis, capillary isoelectric focusing, micellar electrokinetic capillary chromatography, or capillary electrochromatography. In an aspect, the separation is done using capillary gel electrophoresis (CGE), which separates samples by size and detects nucleic acids using a fluorescent dye that binds to the nucleic acids. In an aspect, the separation is done using capillary zone electrophoresis (CZE), which separates samples by electrophoretic mobility, which is directly proportional to the charge to size ratio on the molecule and inversely proportional to the viscosity of the solvent and hydrodynamic radius of the molecule. CZE can be used to separate nucleic acids of different sizes from the intact lentivirus particle (80 to 100 nanometer). The capillary ID for CZE is usually 50 micrometers.

In some aspects, detecting the nucleic acid genome produces a set of corresponding values that can be used to quantify or otherwise analyze the nucleic acid genome. In some aspects, these corresponding values can be plotted on an electropherogram.

The detector can be a UV detector or a fluorescence detector, such as a laser-induced fluorescence (LIF) detector, a lamp-based fluorescence detector, or a native fluorescence detector. The desired quantitation sensitivity will determine the type of detector used. LIF detection offers the benefit of about a 100-fold increase in sensitivity, yet it also requires additional sample manipulation.

In some aspects, the method is used in an amplification-free workflow, a high-throughput screening application, or a rapid screening workflow. The method may also be used to analyze nucleic acid from at least two biomolecules simultaneously, alternatively at least three biomolecules, alternatively at least four biomolecules, alternatively at least five biomolecules, alternatively at least six biomolecules, alternatively at least seven biomolecules, alternatively at least eight biomolecules. The method may also be used for simultaneous analysis of nucleic acid from biomolecules with different genome sizes.

FIG. 3 illustrates a genome integrity analysis for four lentivirus vectors with different genome sizes. RNA from Lentivirus samples at 25 μl in volume and around 1×10⁹ TU in titer were incubated with a chaotropic agent and extracted using solid-phase extraction. Eluted RNA samples were analyzed on a multi-capillary electrophoresis system. The y-axis is relative fluorescence units, and the x-axis is migration time in minutes. Arrows point to intact genome peaks. The pink trace is the RNA size standards with sizes marked in black font, while the orange trace shows the NEB RNA ladder. Electropherograms obtained with LV-ChAT-GFP, LV-CAG-Cre-mCherry, LV-CAG-GFP, and LV-EF1a-GFP-T2A-fluc-Puro are shown in red, green, turquoise, and blue traces with intact RNA genome sizes of 4.5 kb, 7.2 kb, 6.1 kb, and 8.6 kb, respectively. In addition to peaks corresponding to expected intact genome sizes, smaller-sized species were also detected for each LV vector sample. These may be a combination of impurities outside of the LV particles, partial RNA genomes, and smaller RNA fragments inside the LV particles.

Another aspect of the disclosure includes a kit for characterizing genome integrity, wherein the kit comprises a chaotropic agent, a CE capillary, a buffer comprising a polymer matrix, at least one sample solution, and instructions for use. The kit may also include a carrier molecule, a solid-phase extraction cartridge, and/or a digestion enzyme.

### EXAMPLES

### Example 1: Exemplary sample preparation and analysis method

### Materials

Nuclease free water (PN AM9932), SYBR Green II RNA gel stain, 10,000× concentrate in DMSO (PN S7564), RNase-free DNase I (PN Am2222), 10× DNase I buffer (PN AM8170G), and 10× Phosphate Buffered Saline or PBS (PN AM9624) were obtained from Thermo Fisher Scientific, Waltham, MA. Polyvinyl-pyrrolidone (PVP, PN 437190), benzonase (PN E1014-5KU), 0.5 M EDTA, pH 8.0 (PN E7889-100ML), Transcript RNA markers 0.2-10 kb (PN R7020) and 10× Tris Borate EDTA (TBE) buffer (PN 574795), Molecular Biology Grade, Amicon Ultra-0.5 centrifugal filter unit with MW cut off of 100 KDa (PN UFC510024) were from Millipore Sigma, St. Louis, MO. The 5 μm syringe filter (PN 4650) was from PALL Corporation, Port Washington, NY. Rainin LTS filter tips were from Mettler Toledo, Oakland, CA. QIAamp Viral RNA mini Kit (PN 52904) was from Qiagen, Germantown, MD. Lentiviral vectors with titer of around 1×10⁹ transduction units per ml were from SignaGen Laboratories, Rockville, MD. Single stranded RNA ladder 0.5-9 kb (PN N0362S) was from New England BioLabs, Ipswich, MA. Sample Loading Solution (SLS, PN 608082), Pre-assembled, BioPhase BFS Capillary Cartridge (8 capillaries, 30 cm in total length, PN 5080121, FIG. 2) and disposable BioPhase Sample and Reagent Plates (PN 5080311, FIG. 2) were from SCIEX, Framingham, MA. Ethanol (200 proof) was from AAPER Alcohol and Chemical Co., Shelbyville, Kentucky.

### Instrument and Software

A BioPhase 8800 system equipped with a solid-state laser and PMT detector for LIF detection was from SCIEX, Framingham, MA. The excitation wavelength was at 488 nm and emission wavelength at 520 nm, which can be customized as needed. Data acquisition and analysis were performed using BioPhase Software V1.0 (SCIEX, Framingham, MA).

### Preparation of the buffer comprising a polymer matrix

To make 100 ml of the separation buffer, 1 g of PVP was added to 60 ml nuclease free water in a 250 ml glass beaker and allowed to sit at room temperature for about 10 minutes. The solution was swirled gently to help PVP to be completely dissolved. Then, 24 g of urea was added. The solution was mixed with a clean stirring bar for about 20 minutes without heating. After urea was completely dissolved, 10 ml of the 10× TBE buffer was added. After one more minute of stirring, nuclease free water was added until the total volume was 100 ml. The separation buffer contained 1% PVP, 1× TBE, pH 8.3, and 4 M urea. This buffer should be good for one month if stored at 2° C. to 8° C. in 20-30 ml aliquots. Before the sample run, the required amount of gel buffer was warmed up to room temperature and filtered through a 5 μm filter. SYBR Green II dye was added at a 1 to 25,000 dilution.

### Preparation of RNA Ladders

Single-stranded RNA 6000 ladder from NEB was diluted 200 fold with a 1:1 mixture of nuclease-free water and SLS, heated at 70° C. for 2 minutes in a thermal cycler, and then immediately placed on ice for at least 5 minutes. For separation on a BioPhase 8800 system, 50 μl of treated RNA Ladder was transferred to each well on the sample plate before the sequence run. The same conditions were used for the Transcript RNA markers from Sigma except that it was diluted 250 fold.

### Preparation of Lentiviral Vector Samples

RNA was extracted using QIAamp Viral RNA mini Kit. Briefly, 25 μl of each lentiviral vector sample was diluted with 45 μl of 1× PBS and mixed thoroughly 280 μl of the lysis buffer containing 10 ng/ml carrier RNA. After a quick spin, 280 μl of 100% ethanol was added, followed by a thorough mixing and loading of the entire mixture onto the spin column. The column was washed with buffers from the kit. Lentiviral (LV) vector RNA genome sample was eluted with 40 μl of 10×diluted elution buffer (Buffer EB or Buffer AVE) from the kit. Before loading onto the instrument for analysis, 5 μl of the eluted LV vector genome sample was mixed with 20 μl of nuclease-free water and 25 μl of SLS, heated at 70° C. for 2 minutes and immediately cooled on ice for at least 5 minutes. Alternatively, RNA was eluted with 40 μl of nuclease free water, and 20 μl of this sample was mixed with 30 μl SLS before being subjected to the same heat treatment as described for EB eluted samples.

### Benzonase and DNase I Treatments

To remove nucleic acid impurities outside of LV, samples were digested in a 30 μl reaction that contained 25 μl of sample, 1 μl of 1× PBS, 3 μl of 10× DNase I buffer, and 1 μl of benzonase that was diluted 10 fold in 1× DNase I buffer (10 mM Tris-HCl, pH 7.5 at 25° C., 2.5 mM MgCl2, 0.1 mM CaCl2) or 1 μl of DNase I. The digestion was carried out at 37° C. for 30 minutes and terminated by the addition of 3 μl of 50 mM EDTA, followed by heat treatment at 65° C. for 10 minutes.

### Methods and Sequence Creation

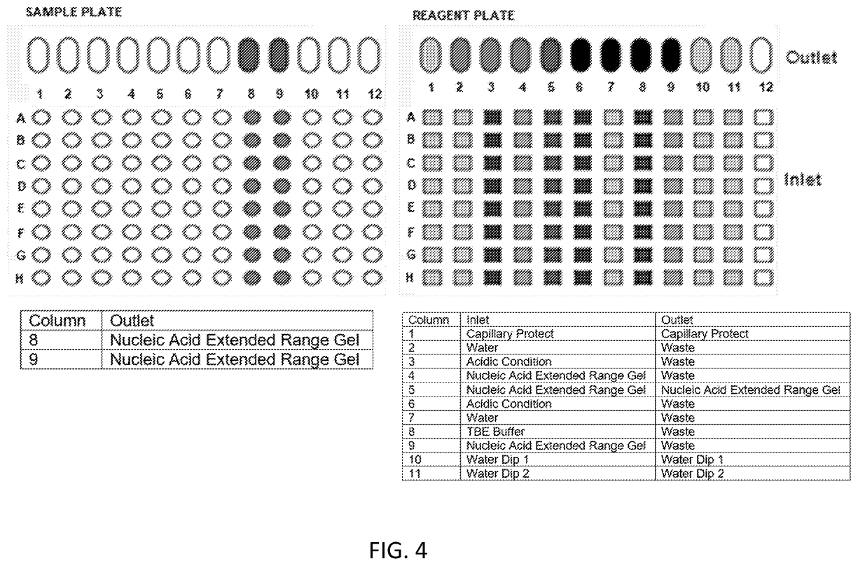

Methods were created using the intuitive tile based drag-and-drop interface in the “Method Editor” module of BioPhase Software, where desired buffers, reagents, specific actions like “rinse” and “inject’ steps were selected to assemble into a method. Similarly, run sequences were created in “Sequence Editor” module of BioPhase Software in which desired methods were selected and applied to each sample column, as described in the BioPhase 8800 system Operator Guide. The amounts of reagents needed were calculated by the BioPhase Software based methods and number of sample injections in the sequence. Once a sequence was created, sample plate layout and reagent plate layout were generated by the BioPhase Software. FIG. 4 shows an example of sample plate (left panel) and reagent plate (right panel) layouts for running 16 samples in two columns. Wells selected were indicated by colored circles on the sample plate, ovals on the outlet plates, and squares on the reagent inlet plate. Tables below the plate layouts provided legends for color codes used in plate layouts.

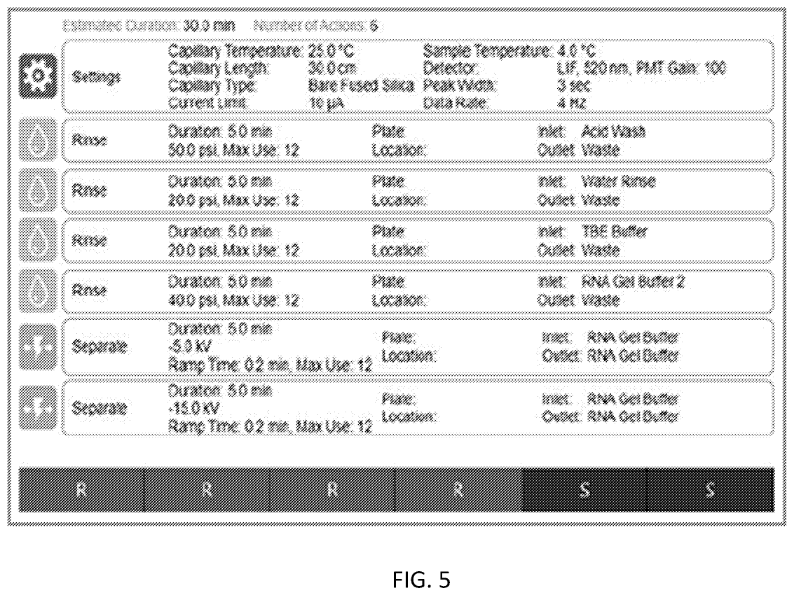

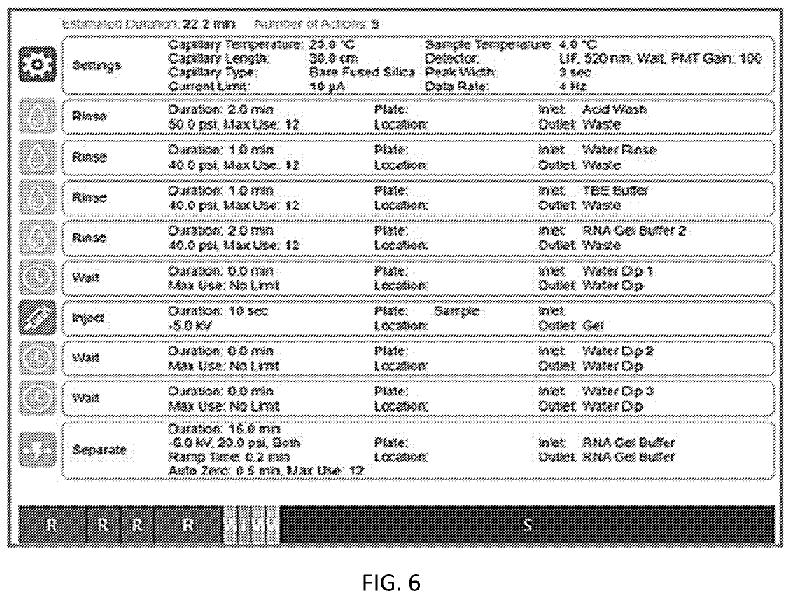

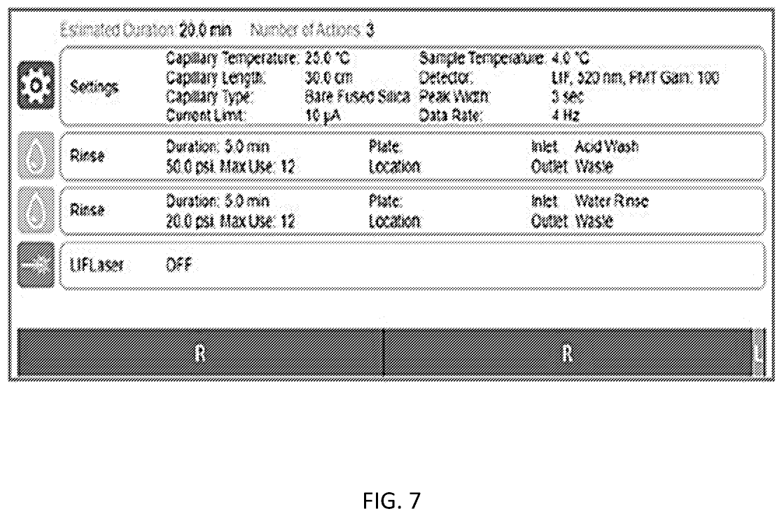

Settings for cartridge conditioning, sample separation, and shutdown methods are provided in FIGS. 5, 6, and 7, respectively.

### Preparation of Sample and Reagent Plates

Recommended fill volumes are shown in table 1.

TABLE 1

| | Sample | Sample | Reagent | |
|---|---|---|---|---|
| Plate | Inlet | Outlet | Inlet | Reagent Outlet |
| Volume | 50 | 1500 | 800 | 2800 (cap protect) |
| per well | | | | 2800 (gel buffer) |
| (μl) | | | | 2800 (water Dip) |
| | | | | 1500 (waste) |

Wells needed to be filled are indicated by the plate layouts as shown in FIG. 4. For the sample and the reagent outlet plates, the reagents were added to indicated wells on the lower side of the plate, away from the chamfered corner. In another experiment, two different elution conditions were compared: elution with nuclease free water or with 10% elution buffer (EB).

### Example 2: Comparison of Different RNA Extraction and Elution Conditions

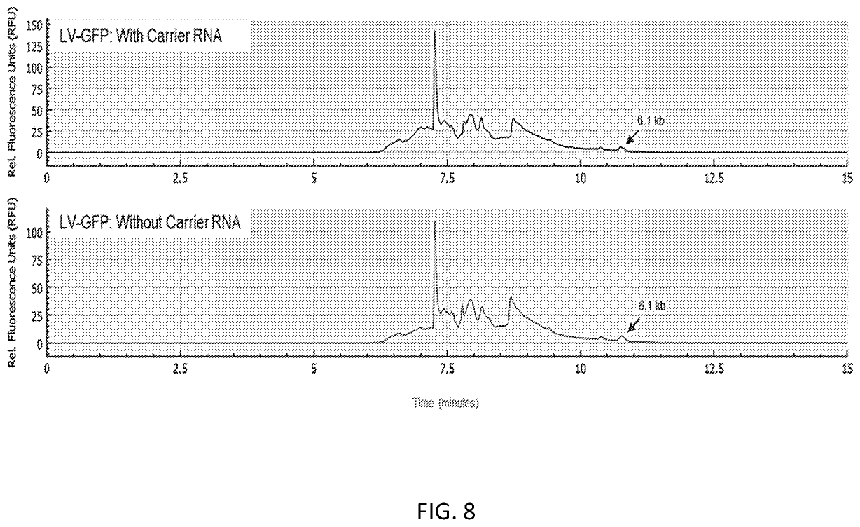

In order to understand the impact of carrier RNA on the yield of RNA extraction, two vials of LV-CAG-GFP samples at 26 μl were pooled, mixed by gentle inverting, and transferred to two Eppendorf tubes at 25 μl each. Both were subjected to the same RNA extraction process with QIAamp Viral RNA Mini Kit, except that one included 10 ng/ml carrier RNA in the lysis buffer and the other without carrier RNA. After elution with 40 μl of nuclease free water, 20 μl of it was mixed with 30 μl SLS, heated at 70° C. for 2 minutes. After a quick chill on ice, samples were loaded on the BioPhase 8800 system for CE-LIF analysis. Results are shown in FIG. 8. The red trace was obtained with RNA extracted with carrier RNA, while the green trace was obtained without carrier RNA. Although the peak profiles were identical in the two traces, the scale in the green trace was smaller, indicating that lower RNA yield was obtained without carrier RNA. Therefore, carrier RNA should be included in the lysis buffer for RNA extraction for maximum yield.

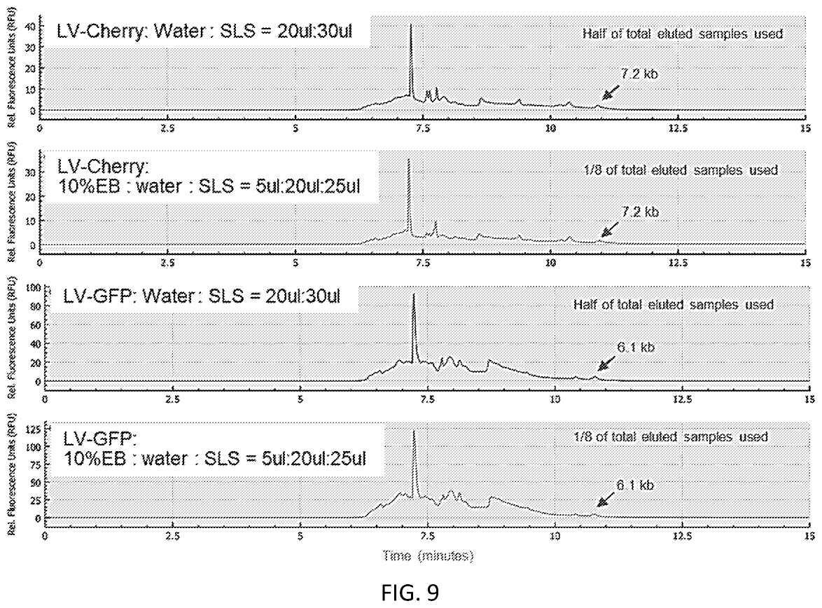

Two vials of LV-CAG-Cre-mCherry were pooled and divided equally before RNA extraction was carried out. One was eluted with 40 μl of nuclease free water, while the other was eluted with 40 μl of 10% EB buffer. Half of the sample eluted with nuclease free water (20 μl) was mixed with 30 μl SLS before heat treatment and CE-LIF analysis while ⅛ (5 μl) of the 10% EB eluted sample was mixed with 20 μl of water and 25 μl of SLS before analysis. The same experiment was done with LV-CAG-GFP. Results are summarized in FIG. 9. Red traces were obtained with eluting RNA with nuclease free water, while Green traces were obtained with RNA eluted with 10% EB. Peak profiles in red and green traces were identical. Although similar signal levels were obtained in both red and green traces, more RNA was detected in samples eluted with 10% EB since only ⅛ of 10% EB eluted sample was analyzed comparing to half of the water eluted sample was analyzed. These results support that 10% EB is a better choice for elution of LV vector RNA from the silicone column.

### Example 3: Effect of Benzonase and DNase I Treatments

In order to determine if some of the small sized peaks detected in FIG. 3 were due to impurities present outside of the LV particles, 25 μl of LV-CAG-Cre-mCherry and LV-CAG-GFP were incubated with Benzonase or RNase-free DNase I or buffer control as described above. RNA was extracted, eluted with 40 μl nuclease-free water. Half of the eluted samples were used for analysis on the BioPhase 8800 system.

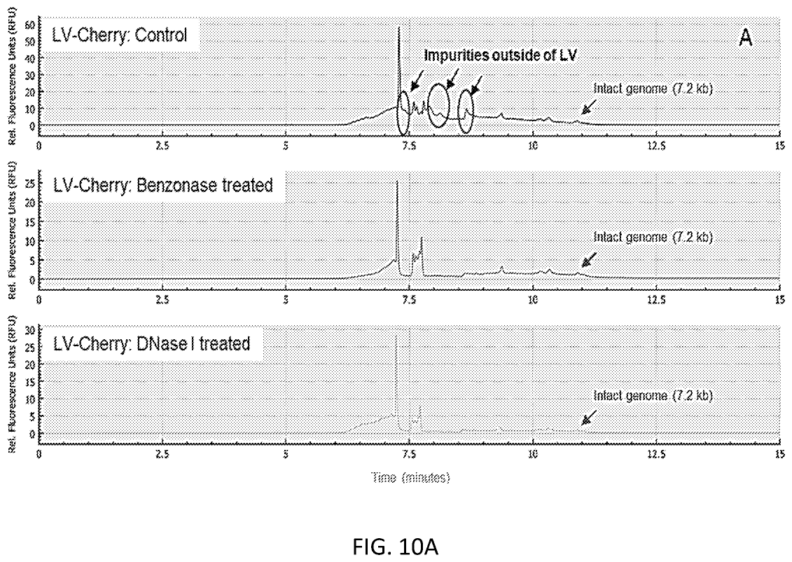

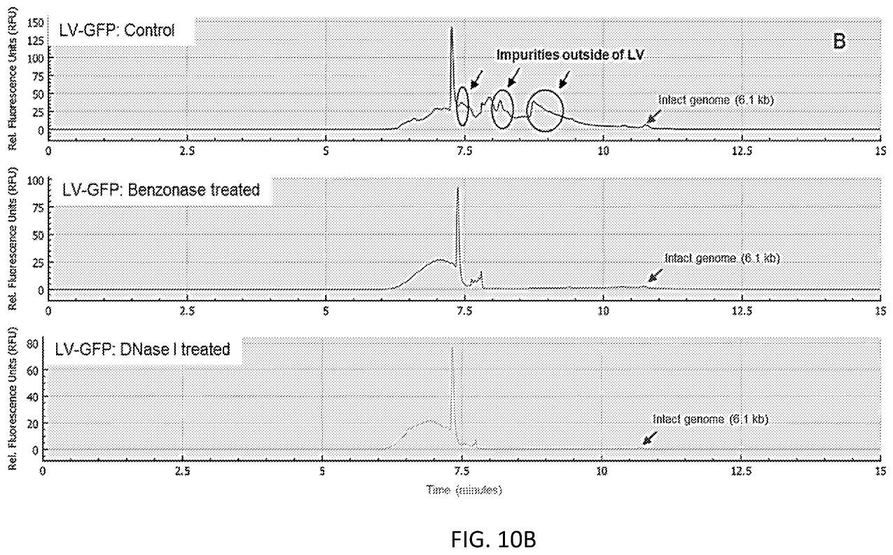

FIGS. 10A and 10B summarize results obtained with LV-CAG-Cre-mCherry (FIG. 10A) and LV-CAG-GFP (FIG. 10B). Red trace is the control, green trace is Benzonase treated, and turquoise trace is DNase I treated. In FIG. 10A, the intact RNA genome peak for LV-CAG-Cre-mCherry was detected in all three traces. The same is true for the peaks located around 7.25 minutes, 7.5 to 8 minutes, 9.4 minutes, and 10.25 minutes, indicating that these species were from the inside of LV particles. The peaks in purple circles were detected in control samples but not in Benzonase or DNase I treated samples, suggesting that these peaks may be from residual DNA contamination present outside of the LV particles. Similarly, the intact genome peak for LV-CAG-GFP and peaks located around 7.25 minutes, 7.5 to 8 minutes, 9.4 minutes, and 10.25 minutes were detected under all three conditions, while peaks in purple circles were only present in the control sample. In addition, there were differences between the Benzonase treated samples and DNase I treated samples around 7.5 to 7.75 minutes area where more peaks were detected in Benzonase treated samples comparing to DNase I treated samples. Although Benzonase and DNase I treatments are useful in identifying the contaminants present outside of the LV particles, further analysis is needed to understand the origin of the species peaks located around 7.25 minutes, 7.5 to 8 minutes, 9.4 minutes, and 10.25 minutes. Peaks located between 7.5 to 8 minutes have estimated sizes around 200 to 300 bases. These peaks could be related to the minus strong-stop cDNA (U5/R region) fragments present within lentiviral capsids as described in published literature.' This minus strong-stop cDNA has been utilized for the quantification of LV vector particle numbers. The minus strong-stop cDNA fragment size for LV-CAG-Cre-mCherry and LV-CAG-GFP is around 250 bases in length, according to the manufacturer.

### Example 4: Repeatability Test for Sample Separation

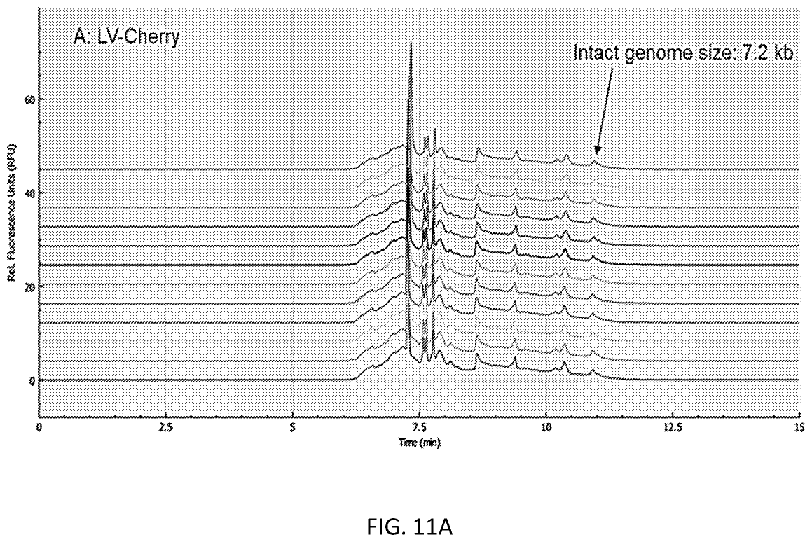

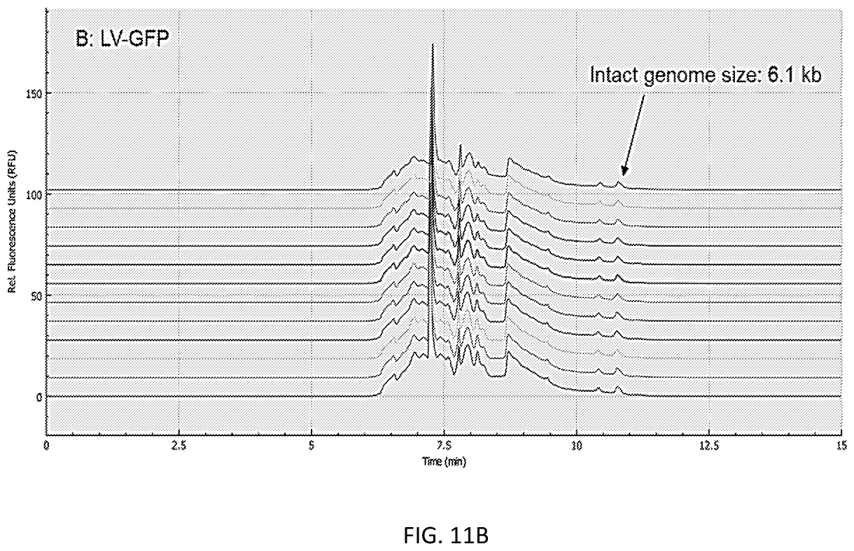

RNA was extracted from 25 μl of LV-CAG-Cre-mCherry and LV-CAG-GFP and eluted with 40 μl of nuclease free water. Half of the eluted sample was mixed with 30 μl of SLS, heat treated, and injected at 5 kv for 10 seconds for 12 consecutive times. As demonstrated in FIGS. 11A and 11B, peak patterns in 12 runs for both LV-CAG-Cre-mCherry and LV-CAG-GFP were very consistent. The CV for migration time for the intact genome peaks was less than 0.5%.

### Example 5: Amplification-Free Workflow for Lentiviral Vector Genome Integrity Analysis

Based on the experiments described above, an amplification-free workflow for lentiviral vector genome integrity analysis is established and shown in FIG. 1.

In this workflow, RNA is extracted directly from viral samples and directly analyzed on the BioPhase 8800 system without further amplification. Although Lentiviral vectors are becoming increasingly used as gene delivery tools, one caveat is their low titer. Consequently, analysis of LV vector RNA genome usually requires amplification by RT-PCR. Both RT and PCR introduce variations. Therefore, the amplification-free workflow developed here for lentiviral vector genome integrity analysis should be very beneficial for rapid and consistent LV vector analysis.

While the present disclosure has been described with reference to certain aspects, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular aspects disclosed, but that the present disclosure will include all aspects falling within the scope of the appended claims.

All patents, patent applications, publications, and descriptions mentioned above are herein incorporated by reference in their entirety.

1. A method for characterizing genome integrity comprising:
incubating at least one biomolecule comprising nucleic acids with a chaotropic agent;
extracting the nucleic acids from the biomolecule;
diluting the nucleic acids;
heating the nucleic acids to denature the nucleic acids;
loading the denatured nucleic acids onto a capillary electrophoresis (CE) capillary, wherein the CE capillary is filled with a buffer comprising a polymer matrix;
applying a separation voltage to the CE capillary to separate the denatured nucleic acids; and detecting at least one nucleic acid genome separated from the denatured nucleic acids with a detector.

2. (canceled)

3. The method of claim 1, wherein the biomolecule is a viral sample, and wherein the viral sample is a viral vector.

4. The method of claim 3, wherein the viral vector comprises a nucleic acid genome, plasmid fragment contamination, and/or DNA fragment contamination from a host cell DNA.

5. The method of claim 3, wherein the viral vector is a retrovirus.

6. The method of claim 3 wherein the viral vector is a lentivirus.

7. The method of claim 6, wherein the lentivirus is a recombinant lentivirus.

8. (canceled)

9. The method of claim 1, further comprising incubating the biomolecule with a carrier molecule.

10. The method of claim 9, wherein the carrier molecule is a poly (A) carrier, a poly (T) carrier, a poly (AT) carrier, glycogen, carrier RNA, or RNA from yeast.

11. The method of claim 9, wherein prior to incubating the biomolecule, the biomolecule is treated with an enzyme.

12. The method of claim 11, wherein the enzyme is a digestion enzyme or is selected from the group consisting of ribonuclease, deoxyribonuclease, endonuclease, and combinations thereof.

13. (canceled)

14. The method of claim 1, wherein the chaotropic agent is a denaturing agent or lysis buffer.

15. The method of claim 1, wherein the chaotropic agent is guanidinium thiocyanate, guanidinium isothiocyanate, n-butanol, ethanol, guanidinium chloride, guanidinium hydrochloride, lithium acetate, magnesium chloride, 2-propanol, sodium dodecyl sulfate, thiourea, urea, sodium iodide, sodium perchlorate, potassium iodide, or combinations thereof.

16. (canceled)

17. The method of claim 1, wherein the nucleic acids are extracted using solid phase extraction, wherein the solid phase extraction is spin column-based purification.

18. (canceled)

19. The method of claim 1, wherein the nucleic acids are diluted using a sample solution, water, or combinations thereof.

20. The method of claim 19, wherein the sample solution is a sample loading solution or formamide.

21-27. (canceled)

28. The method of claim 1, wherein the polymer matrix is selected from the group consisting of crosslinked polymer, linear polymers, slightly branched polymers, linear polyacrylamide, polyethylene oxide, polyethylene glycol, dextran, and polyvinylpyrrolidone.

29. The method of claim 1, wherein the polymer matrix comprises a fluorescent dye.

30-34. (canceled)

35. The method of any one the preceding claims, wherein the method is used in a multi-capillary electrophoresis system workflow, wherein:
the multi-capillary electrophoresis system workflow comprises the use of a cartridge comprising at least two capillaries, alternatively at least three capillaries, alternatively at least four capillaries, alternatively at least five capillaries, alternatively at least six capillaries, alternatively at least seven capillaries, alternatively at least eight capillaries, and/or
wherein the multi-capillary electrophoresis system workflow is used for the simultaneous analysis of nucleic acids from at least two biomolecules, alternatively at least three biomolecules, alternatively at least four biomolecules, alternatively at least five biomolecules, alternatively at least six biomolecules, alternatively at least seven biomolecules, alternatively at least eight biomolecule.

36. (canceled)

37. (canceled)

38. The method of claim 35, wherein at least two of the biomolecules have nucleic acids with different genome sizes.

39. The method of claim 1, wherein the method is used for titer determination of a viral product and/or determination of percent of full capsids in viral product or complete lentiviral particles.

40-48. (canceled)
